(19) United States
(12) Patent Application Publication
(10) Pub. No.: US 2024/0249806 A1
(43) Pub. Date: Jul. 25, 2024

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Takayuki MATSUURA, Tokyo (JP)

(73) Assignee: FUJIFILM Corporation, Tokyo (JP)

(21) Appl. No.: 18/624,095

(22) Filed: Apr. 1, 2024

Related U.S. Application Data
(63) Continuation of application No. PCT/JP2022/036996, filed on Oct. 3, 2022.

(30) Foreign Application Priority Data
Oct. 15, 2021 (JP) 2021-169821

(51) Int. Cl. G16H 10/60; G06F 40/166; G16H 15/00

(52) U.S. Cl. CPC G16H 10/60; G06F 40/166; G16H 15/00

(57) ABSTRACT

An information processing apparatus includes at least one processor. The processor is configured to: acquire first medical document data including first information related to a patient; derive second information, which is different from the first information related to the patient, based on the first information; and generate second medical document data including the derived second information.

This application is a continuation application of International Application No. PCT/JP2022/036996, filed Oct. 3, 2022, the disclosure of which is incorporated herein by reference in its entirety. Further, this application claims priority from Japanese Patent Application No. 2021-169821 filed on Oct. 15, 2021, the disclosures of which is incorporated herein by reference in its entirety.

### BACKGROUND

### 1. Technical Field

The disclosed technology relates to an information processing apparatus, an information processing method, and an information processing program.

### 2. Description of the Related Art

The following technologies are known for processing information related to a specific patient. For example, JP2009-9609A discloses displaying an input screen for progress or the like related to a patient's chief complaint, doctor's findings, and the like, an input screen for prescription or the like related to treatments, drugs, or the like, and an individual input screen for a disease name input screen related to disease names on a display unit, and controlling and storing data, which is input in the screen, in a medical treatment information file.

JP2008-171297A discloses a medical treatment support device including: a sheet file that stores setting information of a sheet input screen for inputting medical treatment information; and a sheet input control unit that has a sheet setting unit, which sets an input item such as a treatment or a prescription on the sheet input screen and controls and stores the setting information in the sheet file, and a medical treatment information storage control unit, which controls and stores the input item selected and input on the sheet input screen in a patient's medical treatment information file.

### SUMMARY

A predetermined medical document in which medical information related to a patient is entered is created immediately before a specific timing from before hospital admission until after hospital discharge of the patient. For example, the medical documents are created immediately before a timing before the hospital admission and after the hospital admission, respectively. The medical document, which is created immediately before a timing after the hospital admission, has an item in which a medical worker enters a result obtained by performing determinations or classifications based on information entered in the previously created medical document. However, at present, since the standards for performing such determinations or classifications are not clear, situations have arisen in which different determinations or classifications are made depending on the work history or knowledge of the medical worker.

The disclosed technology has been made in view of the above points, and the purpose thereof is to support the creation of other medical documents created based on a previously created medical document.

An information processing apparatus according to the disclosed technology is an information processing apparatus comprising at least one processor, in which the processor is configured to: acquire first medical document data including first information related to a patient; derive second information, which is different from the first information related to the patient, based on the first information; and generate second medical document data including the derived second information.

The processor may derive a result, which is obtained by performing determination or classification based on at least one piece of the first information, as the second information.

The processor may derive the second information based on a predetermined rule. Further, the processor may derive the second information by using a trained model that is trained through machine learning, which uses a plurality of combinations of the first information and the second information as training data, that uses the first information as an input, and that uses the second information as an output.

The processor may derive one piece of the second information based on a plurality of pieces of the first information.

The processor may present the second medical document data by associating the first information, which is used for deriving the second information, with the second information.

The processor may receive an input of third information related to the patient and generate the second medical document data in which the second information and the third information are recorded in an identifiable state from each other.

The processor may receive an input of third information related to the patient, generate the second medical document data in which the second information and the third information are recorded, and present the second medical document data such that the second information and the third information are made to be in an identifiable state.

The processor may present, in a case where omission has occurred in at least a part of the first information required for deriving the second information in the first medical document data, omission information, which indicates a fact that the omission has occurred, together with the second medical document data.

The processor may present information, which indicates an item of the first information in which the omission has occurred, as the omission information.

The processor may derive, in a case where omission has occurred in a part of a plurality of pieces of the first information required for deriving the second information, the second information based on another part of the first information in which omission has not occurred, and present the second medical document data, which includes the derived second information, together with the omission information.

The processor may present information, which indicates a fact that accuracy of the second information derived based on the other part of the first information in which the omission has not occurred is relatively low, as the omission information.

The processor may present, in a case where accuracy of the second information derived based on the other part of the first information in which the omission has not occurred is less than a threshold value, information, which indicates an item of the first information in which the omission has occurred, as the omission information, and present, in a case where the accuracy is equal to or greater than the threshold value, information, which indicates a fact that the accuracy of the second information is relatively low, as the omission information.

The first information may be information to be recorded in a plurality of items included in the first medical document data, and the second information may be information to be recorded in at least one item included in the second medical document data. The first medical document data and the second medical document data may be data obtained by converting documents, which are related to hospital admission and discharge of the patient, into data. The second information may be information related to a necessity of nursing care or assistance for the patient.

An information processing method according to the disclosed technology is a method executed by at least one processor included in an information processing apparatus, the method comprises: acquiring first medical document data including first information related to a patient; deriving second information, which is different from the first information related to the patient, based on the first information; and generating second medical document data including the derived second information.

An information processing program according to the disclosed technology is a program causing at least one processor included in an information processing apparatus to execute a process comprising: acquiring first medical document data including first information related to a patient; deriving second information, which is different from the first information related to the patient, based on the first information; and generating second medical document data including the derived second information.

According to the disclosed technology, it is possible to support the creation of another medical document created based on a previously created medical document.

### BRIEF DESCRIPTION OF THE DRAWINGS

Exemplary embodiments according to the technique of the present disclosure will be described in detail based on the following figures, wherein:

FIG. 1 is a diagram showing a function of an information processing apparatus according to an embodiment of the disclosed technology;

FIG. 2A is a diagram showing an example of a schematic content of first medical document data according to the embodiment of the disclosed technology;

FIG. 2B is a diagram showing an example of a schematic content of second medical document data according to the embodiment of the disclosed technology;

FIG. 3 is a diagram showing an example of a hardware configuration of the information processing apparatus according to the embodiment of the disclosed technology;

FIG. 4 is a functional block diagram showing an example of a functional configuration of the information processing apparatus according to the embodiment of the disclosed technology;

FIG. 5 is a diagram showing an example of an aspect in which a derivation unit according to the embodiment of the disclosed technology derives second information for a rule-based determination item;

FIG. 6 is a diagram showing an example of an aspect in which the derivation unit according to the embodiment of the disclosed technology derives second information for an overall determination item;

FIG. 7 is a diagram showing another example of an aspect in which the derivation unit according to the embodiment of the disclosed technology derives second information for the overall determination item;

FIG. 8 is a diagram showing an example of a display form of the second medical document data displayed on a display;

FIG. 9 is a diagram showing an example of a display form of the second medical document data displayed on the display;

FIG. 10 is a diagram showing an example of a display form of the second medical document data displayed on the display;

FIG. 11 is a flowchart showing an example of a flow of a process that is performed by executing a document creation program according to the embodiment of the disclosed technology;

FIG. 12 is a diagram showing an example of a state in which omission occurs in first information input to a determination/classification model according to the embodiment of the disclosed technology;

FIG. 13 is a diagram showing an example of a display form of the second medical document data displayed on the display;

FIG. 14A is a diagram showing an input and output of a first determination/classification model according to the embodiment of the disclosed technology;

FIG. 14B is a diagram showing an input and output of a second determination/classification model according to the embodiment of the disclosed technology;

FIG. 15 is a diagram showing an example of a display form of the second medical document data displayed on the display; and

FIG. 16 is a flowchart showing an example of a flow of a process that is performed by executing the document creation program according to the embodiment of the disclosed technology.

### DETAILED DESCRIPTION

Hereinafter, an example of an embodiment of the disclosed technology will be described with reference to the drawings. The same or equivalent components and portions in the drawings are assigned by the same reference numerals, and the overlapping description will be omitted.

### First Embodiment

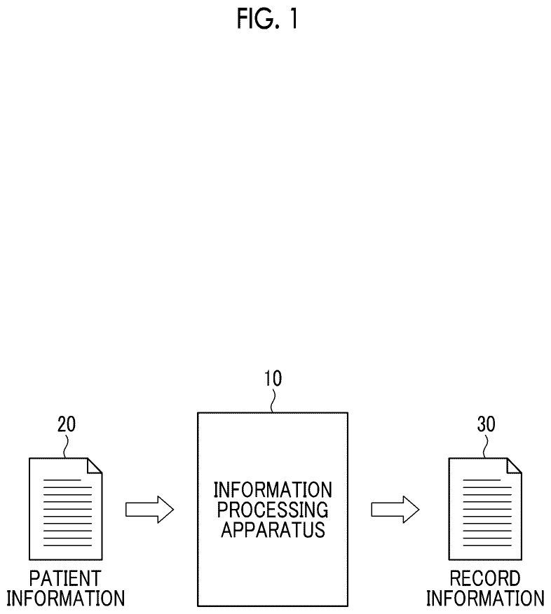

As shown in FIG. 1, first medical document data 20 including medical information related to a patient is input to an information processing apparatus 10 according to an embodiment of the disclosed technology. The information processing apparatus 10 derives medical information, which is different from the above-mentioned medical information related to the patient, based on the medical information included in the first medical document data 20 and generates second medical document data 30 including the derived medical information.

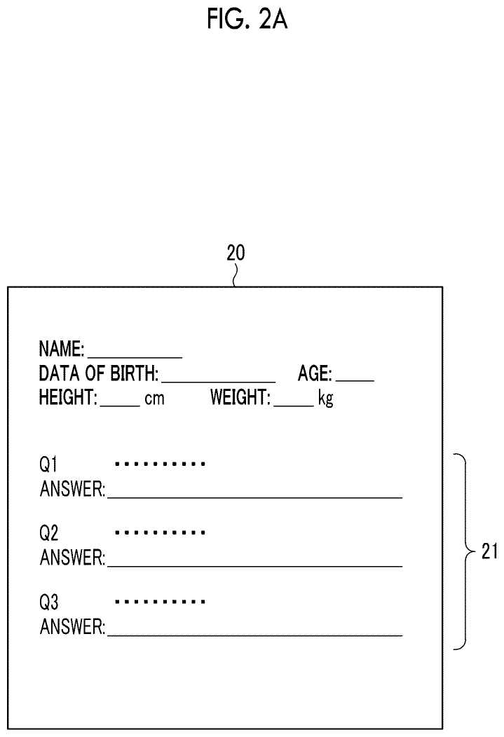

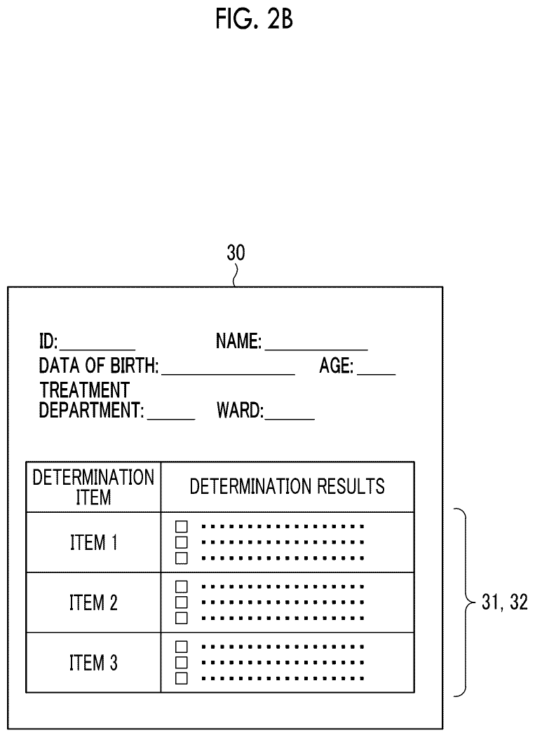

FIG. 2A is a diagram showing an example of a schematic content of the first medical document data 20. The first medical document data 20 may include basic information related to the patient such as a name, a date of birth, an age, a height, and a weight of the patient. Further, the first medical document data 20 may include, as the medical information related to the patient, information related to physical and mental of the patient (disease/medical history, medication status, presence or absence of dementia, presence or absence of behavior such as wandering or the like), information related to the living environment (family composition, living history, caregiver's care method, family caregiver's status, or the like), and information related to activities of daily living that can be carried out by the patient and activities of living that require nursing care or assistance. Hereinafter, the medical information included in the first medical document data 20 is referred to as first information 21. The first information 21 may be recorded for each item, for example, as an answer to a plurality of questions expressed as Q1, Q2, in FIG. 2A. That is, the first information 21 may be information to be recorded in a plurality of items included in the first medical document data 20. Further, the first information 21 may be recorded by checking a check box corresponding to each item. Further, the first information 21 may be recorded by inputting a text for each item. Further, for example, various measurement values, which are obtained through a blood test, a urine test, or the like, may be recorded as the first information 21.

Although the first medical document data 20 is not particularly limited, for example, the first medical document data 20 may be data obtained by converting medical documents, which are related to hospital admission and discharge of the patient, into data or may be data obtained by converting information sheet at the time of hospital admission (or information coordination sheet at the time of hospital admission) or a medical document, which is created for a similar purpose of the information sheet at the time of hospital admission, into data. For example, the information related to physical and mental of the patient (disease/medical history, medication status, presence or absence of dementia, presence or absence of behavior such as wandering or the like), the information related to the living environment (family composition, living history, caregiver's care method, family caregiver's status, or the like), and information related to activities of daily living that can be carried out by the patient and activities of living that require assistance, are entered in the information sheet at the time of hospital admission. The purpose of the information sheet at the time of hospital admission, in a case where a person living at home is hospitalized, is to confirm the coordination between the home care team and the medical institution, to smoothly promote in-hospital care and hospital discharge support/home care transition support, and to help the patient continue living in the community and improve patient's quality of life (QOL) by local comprehensive support centers or home care support offices, which are already involved in home care, informing the medical institution of the patient's physical and mental condition, living environment, or the like. The information sheet at the time of hospital admission is created by a care manager or the like who holds an interview with the patient, the patient's family, or these people immediately before a timing of the hospital admission. The first medical document data 20 may be generated by converting the medical document as exemplified above into data by using a known character recognition technology such as optical character recognition (OCR).

FIG. 2B is a diagram showing an example of a schematic content of the second medical document data 30. The second medical document data 30 may include, for example, basic information related to the patient such as an identification (ID) for identifying the patient, a name, a date of birth, and an age of the patient. Further, the second medical document data 30 may include, for example, information related to a treatment department or a ward in which the patient is hospitalized.

Further, the second medical document data 30 may include, for example, information related to a main disease name at the time of hospital admission, type of hospital admission (emergency hospital admission, re-hospital admission, repeated hospital admissions and discharges), residence before the hospital admission (hospital, facility), and a necessity of nursing care or assistance in activities of daily living (ADL), as medical information different from the first information 21 related to the patient. Among these pieces of medical information included in the second medical document data 30, the information to be recorded through an automatic input by the information processing apparatus 10 is hereinafter referred to as second information 31, and information to be recorded through a manual input by a user is hereinafter referred to as third information 32. In the present embodiment, among the plurality of items included in the second medical document data 30, an item (that is, an item of which information is recorded through an automatic input) in which the second information 31 is recorded and an item (that is, an item of which information is recorded through a manual input) in which the third information 32 is recorded are predetermined items. As illustrated in FIG. 2B, the second information 31 and the third information 32 may be recorded by checking a corresponding check box for each item. Further, the second information 31 and the third information 32 may be recorded by inputting a text for each item.

Although the second medical document data 30 is not particularly limited, the second medical document data 30 may be data obtained by converting the medical documents, which are related to hospital admission and discharge of the patient, into data or may be data obtained by converting a hospital discharge support risk screening sheet or a medical document, which is created for a similar purpose of the hospital discharge support risk screening sheet, into data. In this case, the second information 31 and the third information 32 may include information related to a necessity of nursing care or assistance for the patient. For example, the information related to the main disease name at the time of hospital admission, the type of hospital admission (emergency hospital admission, re-hospital admission, repeated hospital admissions and discharges), the residence before the hospital admission (hospital, facility), and a necessity of nursing care or assistance in activities of daily living (ADL) are entered in the hospital discharge support risk screening sheet. The purpose of the hospital discharge support risk screening sheet is to evaluate whether or not hospital discharge support/hospital discharge adjustment is needed for the patient/family immediately after the patient is hospitalized and to clarify the support needed for transitioning from a place of treatment to a place of living. The hospital discharge support risk screening sheet is created by a medical worker at a patient hospital at a relatively early time (for example, within 3 days after the hospital admission) after the hospital admission of the patient. The second medical document data 30 is created at a time point later than a creation time point of the first medical document data 20.

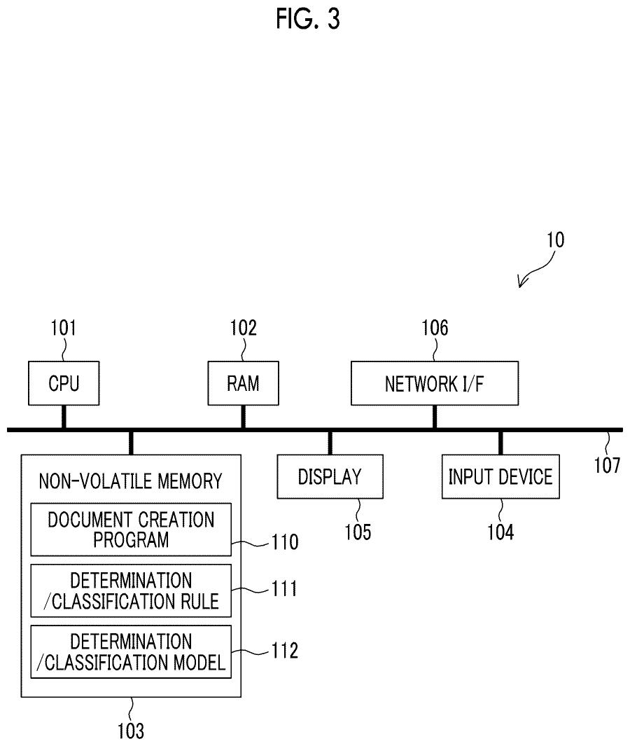

FIG. 3 is a diagram showing an example of a hardware configuration of the information processing apparatus 10. The information processing apparatus 10 includes a central processing unit (CPU) 101, a random access memory (RAM) 102, a non-volatile memory 103, an input device 104 including a keyboard, a mouse, and the like, a display 105, and a network interface 106. These pieces of hardware are connected to a bus 107.

For example, the display 105 is a liquid crystal display or a light emitting diode (LED) display. Further, the input device 104 may be any one of a near input device, such as a touch panel display, a voice input device, such as a microphone, or a contactless input device, such as a camera or a sensor. The network interface 106 is an interface for connecting the information processing apparatus 10 to a network. The non-volatile memory 103 is a storage medium, such as a hard disk and a flash memory. A document creation program 110, a determination/classification rule 111, and a determination/classification model 112 are stored in the non-volatile memory 103. The RAM 102 is a work memory for the CPU 101 to execute a process. The CPU 101 loads the document creation program 110 stored in the non-volatile memory 103 to the RAM 102 and executes a process according to the document creation program 110. The CPU 101 is an example of a “processor” in the disclosed technology. The determination/classification rule 111 is an example of a “predetermined rule” in the disclosed technology. The determination/classification model 112 is an example of a “trained model” according to the disclosed technology.

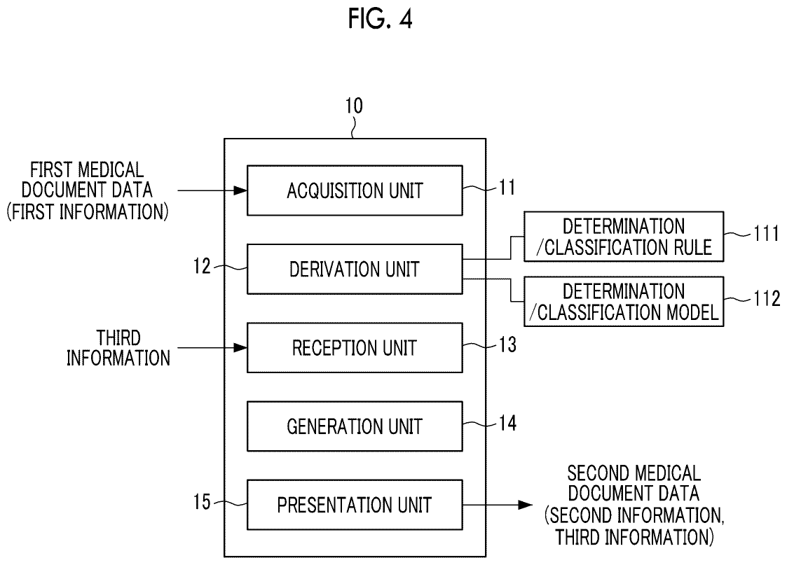

FIG. 4 is a functional block diagram showing an example of a functional configuration of the information processing apparatus 10. The information processing apparatus 10 includes an acquisition unit 11, a derivation unit 12, a reception unit 13, a generation unit 14, and a presentation unit 15. The information processing apparatus 10 functions as the acquisition unit 11, the derivation unit 12, the reception unit 13, the generation unit 14, and the presentation unit 15 by the CPU 101 executing the document creation program 110.

The acquisition unit 11 acquires the first medical document data 20 including the first information 21. First medical document corresponding to the first medical document data 20 is created by, for example, a care manager who holds an interview with the patient, the patient's family, or these people immediately before a timing of the hospital admission, for example. The first medical document data 20 is generated by converting the first medical document into data.

The derivation unit 12 derives second information 31 different from the first information 21 related to the patient, based on the first information 21 included in the first medical document data 20. The derivation unit 12 derives a result obtained by performing determination or classification based on the first information 21, as the second information 31.

Here, the second information 31 includes information derived by performing determination or classification based on at least one piece of first information 21 and the predetermined rule and information derived by performing overall determination or classification based on knowledge related to a plurality of pieces of first information 21. Hereinafter, in the second medical document data 30, among the plurality of items in which the second information 31 is recorded, an item in which the former case is recorded is referred to as a “rule-based determination item” and an item in which the latter case is recorded is referred to as an “overall determination item”. In the present embodiment, an assumption is made that it is determined in advance whether each item included in the second medical document data 30 is a rule-based determination item or an overall determination item. Further, in each of the rule-based determination item and the overall determination item, the first information 21 to be referred to for deriving the second information 31 is predetermined.

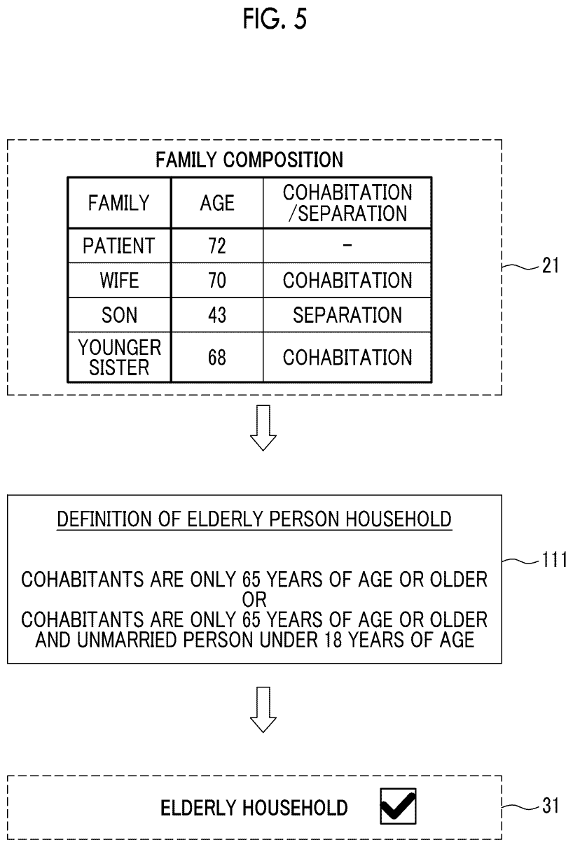

The derivation unit 12 derives the second information 31 by using the determination/classification rule 111 stored in the non-volatile memory 103 for the rule-based determination item. The determination/classification rule 111 is data for defining a rule that is applied to perform determination or classification based on the first information 21. FIG. 5 is a diagram showing an example of an aspect in which the derivation unit 12 derives the second information 31 for a rule-based determination item. FIG. 5 illustrates a case in which second information 31, which indicates whether or not the patient is from an elderly household, is derived based on the first information 21 indicating the family composition of the patient and the determination/classification rule 111 that defines a “definition of elderly person household”. According to the determination/classification rule 111 illustrated in FIG. 5, the elderly person household is defined as “cohabitants are only 65 years of age or older, or cohabitants are only 65 years of age or older and unmarried person under 18 years of age”. The derivation unit 12 derives the second information 31 indicating that the patient is from an elderly household, based on the first information 21 that indicates the family composition of the patient and the determination/classification rule 111 that defines a “definition of elderly person household” illustrated in FIG. 5. As another example in a case where the rule-based determination is performed, there is a case where the second information 31 indicating the age of the patient is derived based on the first information 21 indicating the date of birth of the patient.

On the other hand, the derivation unit 12 derives the second information 31 by using the determination/classification model 112 stored in the non-volatile memory 103 for the overall determination item. The determination/classification model 112 is a trained model that is trained through machine learning, which uses a plurality of combinations of the first information 21 and the second information 31 as training data, that uses the first information 21 as an input, and that uses the second information 31 as an output. It is preferable that the determination/classification model 112 is built by using combinations of the first information 21 and the second information 31, which are obtained for a plurality of patients, as training data. Accordingly, various cases are covered in the training data, and the accuracy of determination or classification in the determination/classification model 112 can be improved.

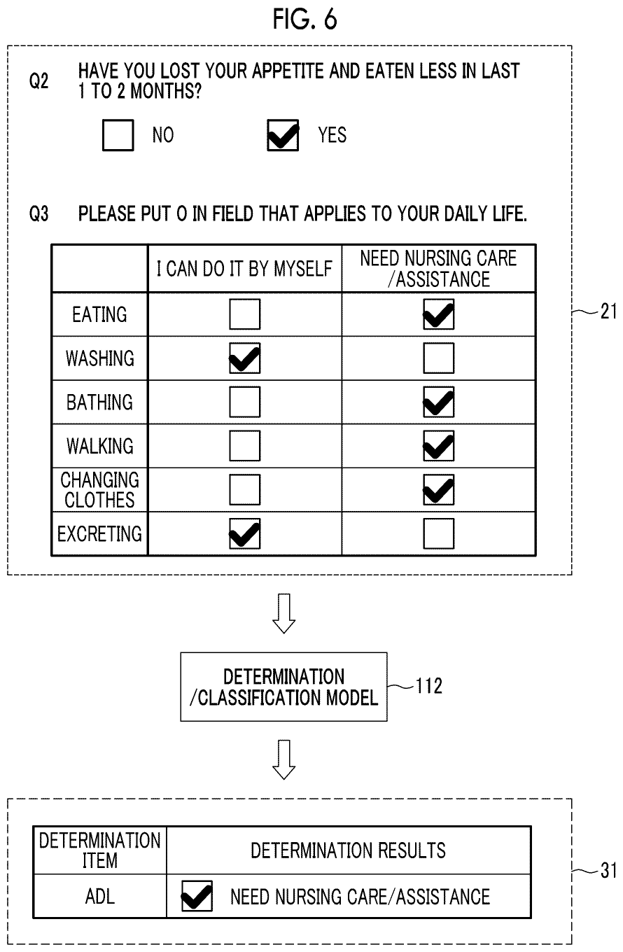

FIG. 6 is a diagram showing an example of an aspect in which the derivation unit 12 derives the second information 31 regarding the overall determination item. In FIG. 6, a case is illustrated in which the first information 21, which indicates the patient's recent eating status and the necessity of nursing care or assistance in activities of daily living, is input to the trained determination/classification model 112, and the determination/classification model 112 derives the second information 31, which indicates the necessity of nursing care or assistance regarding the patient's overall ADL.

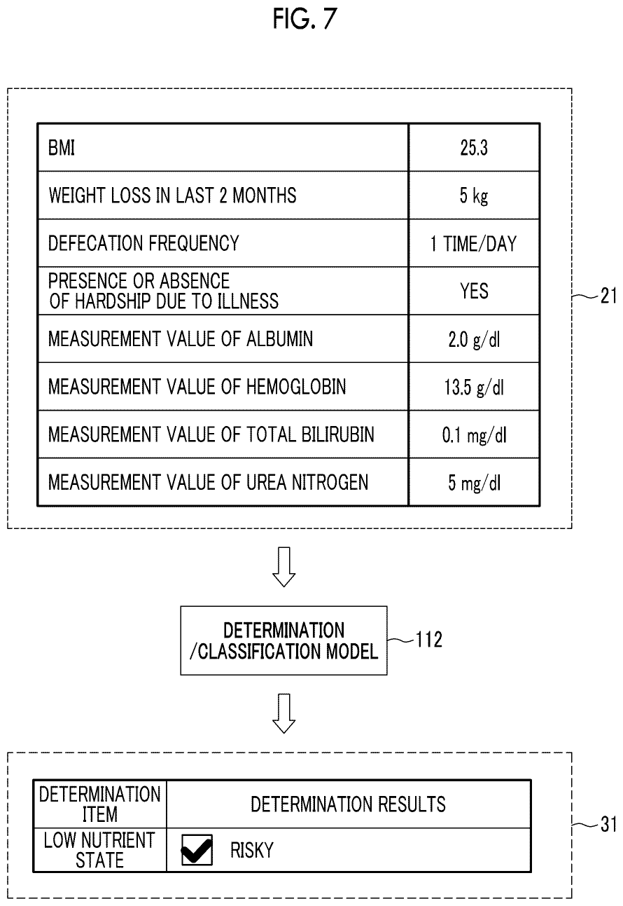

FIG. 7 is a diagram showing another example of an aspect in which the derivation unit 12 derives the second information 31 for the overall determination item. In FIG. 7, a case is illustrated in which the first information 21, which indicates the patient's body mass index (BMI), recent weight loss, defecation frequency, presence or absence of hardship due to illness, and various measurement values, is input to the trained determination/classification model 112, and the determination/classification model 112 derives the second information 31, which indicates the presence or absence of a risk of the patient becoming a low nutrient state. As illustrated in FIGS. 6 and 7, the derivation unit 12 derives one piece of second information 31 based on the plurality of pieces of first information 21 for the overall determination item. In other words, the determination/classification model 112 is a model in which the plurality of pieces of first information 21 are used as inputs, and one piece of second information 31 is used as an output.

The reception unit 13 receives an input of the third information 32 to be recorded through a manual input from the user in the second medical document data 30. The reception unit 13 may prompt the user to input the third information 32 by displaying, on the display 105, a content of a manual input item in which the third information 32 is recorded. The user can input the third information 32 by using the input device 104. The reception unit 13 receives the third information 32 input by the user.

The generation unit 14 generates the second medical document data 30 including the second information 31, which is derived by the derivation unit 12, and the third information 32, which is received by the reception unit 13. A format of the second medical document data 30 is predetermined, and the generation unit 14 generates the second medical document data 30 by recording the corresponding second information 31 or third information 32 in each item of the second medical document data 30. The generation unit 14 may record the second medical document data 30 by diversion from the first medical document data 20 for the same information (for example, a name, a date of birth, an age, or the like of the patient) as the information recorded in the first medical document data 20 among the information to be recorded in the second medical document data 30.

The presentation unit 15 presents the second medical document data 30 generated by the generation unit 14. The presentation of the second medical document data 30 may be performed, for example, by displaying the second medical document data 30 on the display 105 or may be performed by outputting a second medical document corresponding to the second medical document data 30 from a printer (not shown) in response to a request of the user.

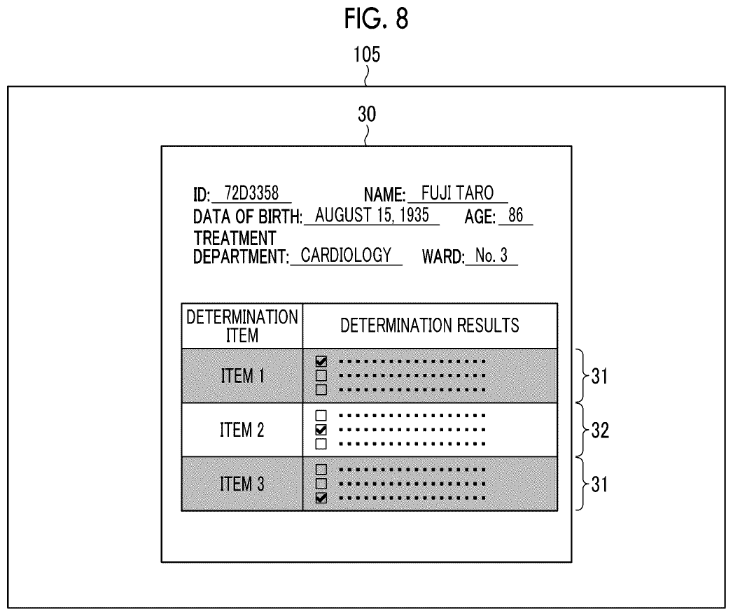

FIG. 8 is a diagram showing an example of a display form of the second medical document data 30 displayed on the display 105. As shown in FIG. 8, the presentation unit 15 displays the second medical document data 30 on the display 105 such that the second information 31 and the third information 32 are in an identifiable state from each other. FIG. 8 illustrates an aspect in which background colors of a record field of the second information 31 and a record field of the third information 32 are different from each other. In this example, the background color of the record field of the second information 31 derived by the derivation unit 12 is displayed in gray, and the background color of the enter field of the third information 32 recorded through a manual input by the user is displayed in white. In this way, by displaying the second information 31 and the third information 32 in a state of being distinguishable from each other, it becomes possible to easily understand whether the information, which is recorded in the second medical document data 30, is recorded through an automatic input by the information processing apparatus 10 or is recorded through a manual input by the user. Accordingly, work of checking the appropriateness for the second information 31 recorded through an automatic input, is facilitated.

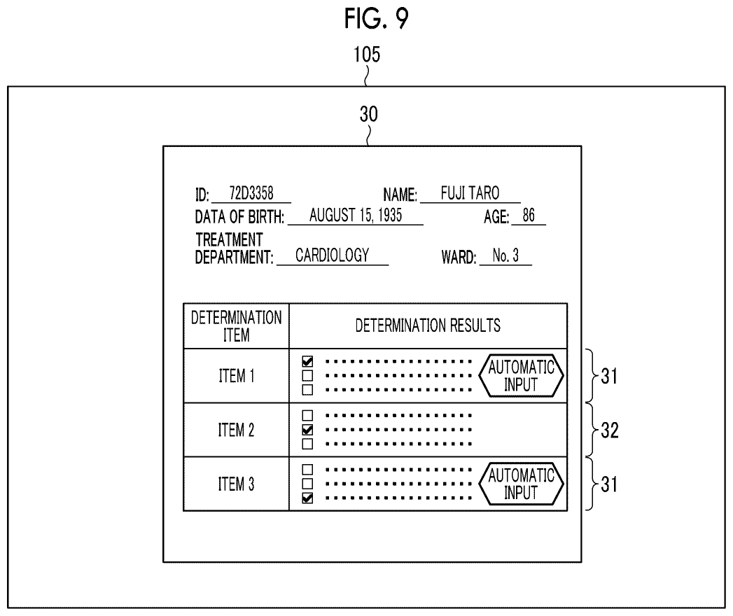

The presentation unit 15 may display the second information 31 and the third information 32 with check marks or text in different colors. Further, as illustrated in FIG. 9, the second information 31 may be displayed with an icon indicating that the second information 31 is recorded through an automatic input.

Further, the generation unit 14 may generate the second medical document data 30 in which the second information 31 and the third information 32 are identifiable as illustrated in FIGS. 8 and 9, and then the presentation unit 15 may present the second medical document data 30 generated by the generation unit 14, or the generation unit 14 may supply additional information, which indicates whether the information included in the second medical document data 30 is the second information 31 or the third information 32, to the presentation unit 15 together with the second medical document data 30, and then the presentation unit 15 may display the second information 31 and the third information 32 in an identifiable manner according to the additional information as illustrated in FIGS. 8 and 9 in a case where the second medical document data 30 is presented.

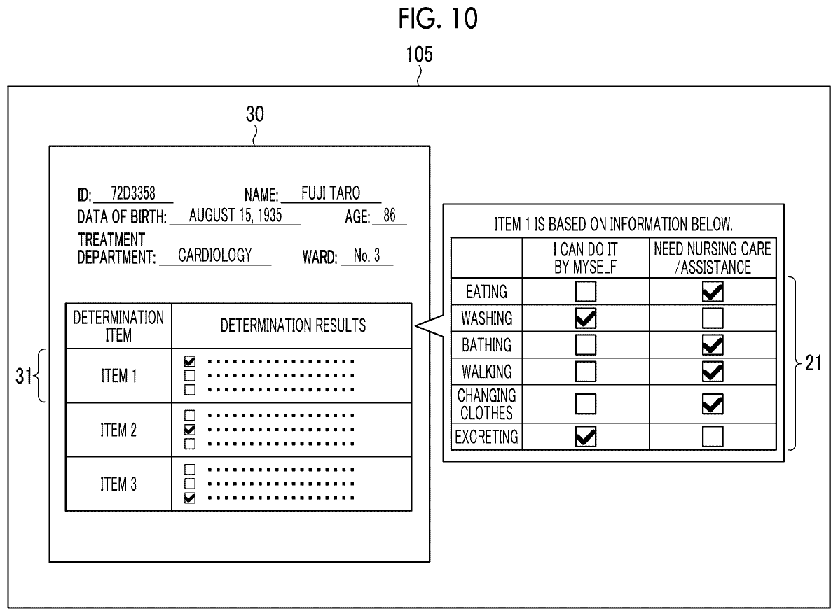

Further, as illustrated in FIG. 10, the presentation unit 15 may present the second medical document data 30 by associating the first information 21, which is used for deriving the second information 31, with the corresponding second information 31. In the example shown in FIG. 10, regarding Item 1 in which the second information 31 of the second medical document data 30 is recorded, the first information 21, which is the basis for deriving the corresponding second information 31, is displayed on the right side of Item 1. By performing such basis display, work of checking the appropriateness for the second information 31 derived by the derivation unit 12, is facilitated. A format of a field where the first information 21 is presented is not limited thereto. A display position of the first information 21 is not limited thereto as long as a correspondence relationship with the second information 31, which is derived based on the first information 21, is clear. For example, in a case where Item 1 is the second information 31, the first information 21, which is used as a basis for deriving the second information 31, may be displayed between Item 1 and Item 2. Further, the first information 21, which is used as a basis for deriving the second information 31, may be always displayed in a case of presenting the second medical document data 30, and in a case where the second information 31 is designated, the first information 21, which is a basis for deriving the second information 31, may be displayed as a pop-up. The designation of the second information 31 may be performed by, for example, receiving an input from the user with respect to a display field of an item, an automatic input icon, or the like by the input device 104.

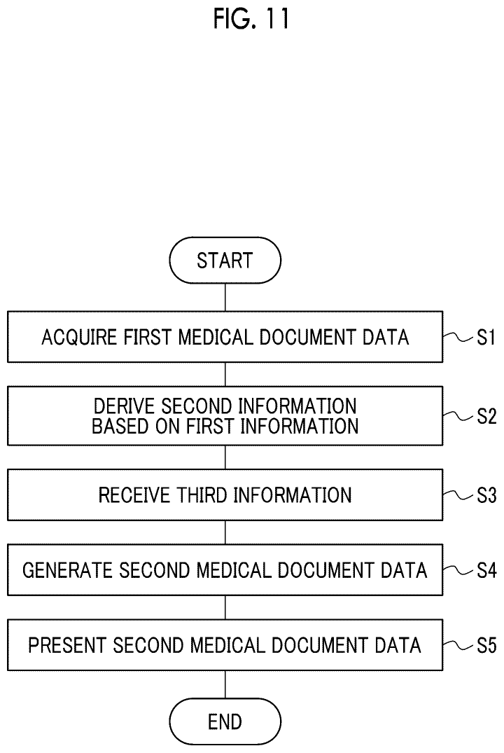

FIG. 11 is a flowchart showing an example of a flow of a process that is performed by executing the document creation program 110 by the CPU 101. The document creation program 110 is executed, for example, in a case where the user issues an instruction to start the process by operating the input device 104.

In step S1, the CPU 101 functions as the acquisition unit 11 and acquires the first medical document data 20 including the first information 21 related to the patient.

In step S2, the CPU 101 functions as the derivation unit 12 and derives the second information 31, which is different from the first information 21, related to the patient, based on the first information 21 included in the first medical document data 20 acquired in step S1. The CPU 101 derives the second information 31 based on the determination/classification rule 111 stored in the non-volatile memory 103 for the rule-based determination item in the second medical document data 30. Further, the CPU 101 derives the second information 31 by using the determination/classification model 112 stored in the non-volatile memory 103 for the overall determination item in the second medical document data 30.

In step S3, the CPU 101 functions as the reception unit 13 and receives an input of the third information 32 recorded in the second medical document data 30 through a manual input. In step S4, the CPU 101 functions as the generation unit 14 and generates the second medical document data 30 including the second information 31 derived in step S2 and the third information 32 received in step S3.

In step S5, the CPU 101 functions as the presentation unit 15 and presents the second medical document data 30, which is generated in step S4, for example, by displaying the second medical document data 30 on the display 105. The presentation unit 15 displays the second medical document data 30 such that the second information 31, which is derived by the derivation unit 12, and the third information 32, which is recorded through a manual input, are in an identifiable state from each other.

As described above, the information processing apparatus 10 according to the present embodiment acquires the first medical document data 20 including the first information 21 related to the patient, derives the second information 31, which is different from the first information 21 related to the patient, based on the first information 21, and generates the second medical document data 30 including the derived second information 31. The information processing apparatus 10 derives a result obtained by performing determination or classification based on at least one piece of the first information 21, as the second information 31.

According to the information processing apparatus 10 of the present embodiment, in a case where the second information 31 is derived, it is possible to avoid a situation where different determinations or classifications are made depending on the work history or knowledge of the medical worker. That is, according to the information processing apparatus 10 of the present embodiment, it is possible to support the creation of another medical document created based on the previously created medical document.

In the above description, although a case has been exemplified in which the first medical document data 20 corresponds to the information sheet at the time of hospital admission (or the information coordination sheet at the time of hospital admission), and the second medical document data 30 corresponds to the hospital discharge support risk screening sheet, the disclosed technology is not limited to this aspect. The first medical document data 20 may correspond to the hospital discharge support risk screening sheet, and the second medical document data 30 may correspond to a hospital discharge support plan document. The purpose of the hospital discharge support plan document is to support the patient being discharged from the hospital with peace of mind and satisfaction and to support the patient such that he or she can continue to receive medical treatment and maintain a lifestyle in a familiar area at an early stage.

In a case where the second medical document data 30 corresponds to the hospital discharge support plan document, examples of the second information 31 recorded in the second medical document data 30 include information indicating the necessity of continuation of rehabilitation. In this case, examples of the plurality of pieces of first information 21 used for deriving the second information 31 include information indicating a patient's age, the purpose of hospital admission, a nursing care insurance certification standard, a necessity of assistance in walking, a necessity of assistance in bathing, whether a patient lives alone or not, whether a patient was using a wheelchair at the stage of hospital admission, and whether a patient was using a cane at the stage of hospital admission.

Further, another example of the second information 31 in a case where the second medical document data 30 corresponds to the hospital discharge support plan document includes information indicating whether an expected hospital discharge destination is home. In this case, examples of the plurality of pieces of first information 21 used for deriving the second information 31 include information indicating a patient's age, the purpose of hospital admission, a nursing care insurance certification standard, whether a patient has forgetfulness that interferes with daily life, and whether medical devices (colostomy, blood sugar-related device, oxygen-related device) are used. The first information 21 exemplified above is information that is acquired from the information sheet at the time of hospital admission or the hospital discharge support risk screening sheet created at a time point before the creation time point of the hospital discharge support plan document. Further, the disclosed technology can also be applied to a case of creating medical documents other than the document related to the hospital admission and discharge of the patient.

### Second Embodiment

The information processing apparatus 10 according to a second embodiment of the disclosed technology includes a function of presenting, in a case where omission has occurred in at least a part of the first information 21 required for deriving the second information 31 in the first medical document data 20, omission information, which indicates a fact that the omission has occurred, together with the second medical document data.

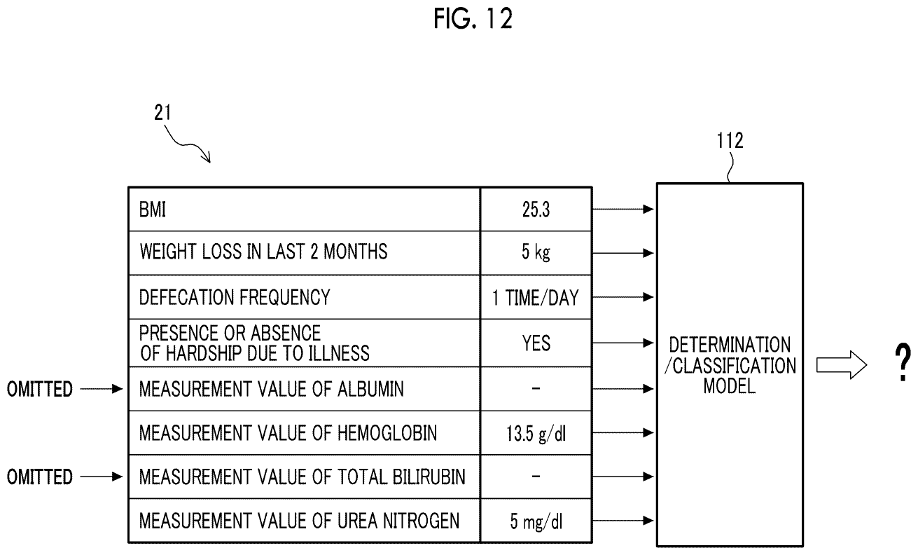

In FIG. 12, a case is illustrated in which some of the measurement values (measurement value of albumin and measurement value of bilirubin) that are the necessary first information 21 are omitted in a case where the first information 21, which indicates the patient's body mass index (BMI), recent weight loss, defecation frequency, presence or absence of hardship due to illness, and various measurement values, is input to the trained determination/classification model 112, and the determination/classification model 112 derives the second information 31, which indicates the presence or absence of a risk of the patient becoming a low nutrient state.

In a case where the determination/classification model 112 is a model trained by using all the pieces of first information 21 required for deriving the second information 31, as shown in FIG. 12, the determination/classification model 112 cannot derive the second information 31 in a case where at least part of the first information 21 is omitted. In this case, the generation unit 14 generates the second medical document data 30 in which the second information 31 is defined as unrecorded for the corresponding item.

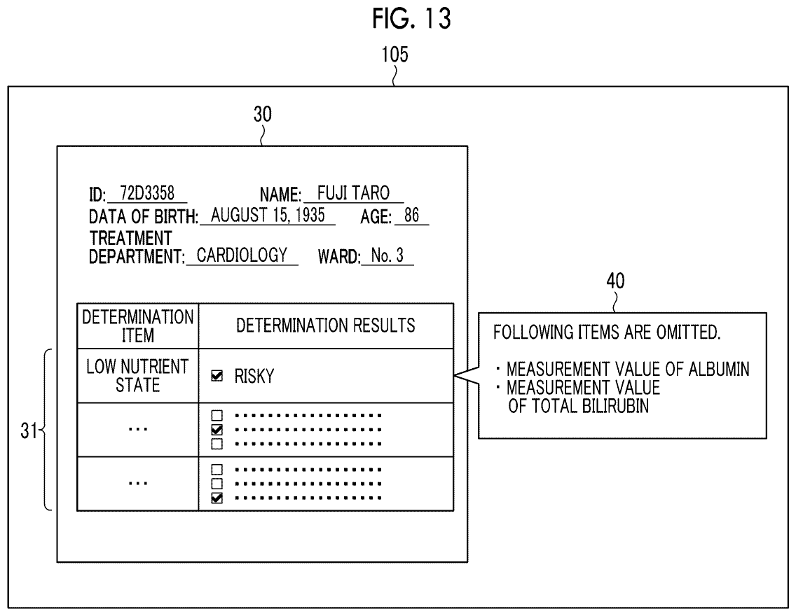

As illustrated in FIG. 13, the presentation unit 15 displays the second medical document data 30, in which the second information 31 is defined as unrecorded for the corresponding item, on the display 105 together with omission information 40. FIG. 13 illustrates the second medical document data 30 in which a checkbox indicating the presence or absence of the risk of becoming a low nutrient state is left blank. This means that the second information 31 (that is, information related to the presence or absence of the risk of becoming a low nutrient state) cannot be derived due to the omission of the first information 21. Further, FIG. 13 illustrates a case where information, which indicates items (measurement value of albumin and measurement value of bilirubin) of the first information 21, in which omission has occurred, is displayed as the omission information 40 in association with the corresponding items. The unrecorded second information 31 and the second information 31 derived by using the determination/classification model 112 may be displayed in different display aspects. For example, similar to the second information 31 and the third information 32 in FIG. 8, the background colors of target items may be different between the unrecorded second information 31 and the second information 31 derived by using the determination/classification model 112. Further, the unrecorded second information 31 may be presented with an icon described as unrecorded instead of the automatic input icon displayed in FIG. 9. Further, similar to the field in which the first information 21 is presented in FIG. 10, a display format of the omission information 40 is not limited thereto. A display position of the omission information 40 is not limited as long as the correspondence relationship with the unrecorded second information 31 is clear through the omission information 40. For example, in a case where Item 1 is the unrecorded second information 31, the omission information 40 corresponding to the unrecorded second information 31 may be displayed between Item 1 and Item 2. Further, the omission information 40 may be always displayed in a case where the second medical document data 30 is presented, or the omission information 40 corresponding to the unrecorded second information 31 may be displayed as a pop-up in a case where the unrecorded second information 31 is designated. The designation of the unrecorded second information 31 may be performed by, for example, receiving an input from the user with respect to a display field of an item, an automatic input icon, or the like by the input device 104.

In the above description, although a case has been exemplified in which the second information 31 cannot be derived in a case where omission has occurred in a part of the first information 21, it is also possible to configure the information processing apparatus 10 to derive the second information 31 even in a case where omission has occurred in a part of the first information 21. That is, in a case where omission has occurred in a part of the plurality of pieces of first information 21 required for deriving the second information 31, the derivation unit 12 derives the second information 31 based on the other part of the first information 21 in which omission has not occurred.

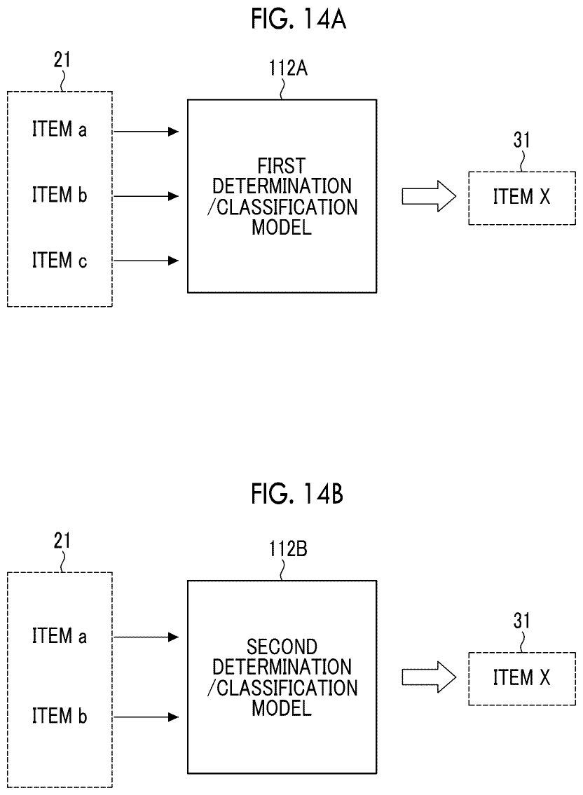

For example, determination/classification models may be used according to the omitted items of the first information 21 by creating a plurality of determination/classification models in which the number and combination of items of the first information 21 to be input are different from each other. Specifically, a first determination/classification model 112A, in which Item a, Item b, and Item c of the first information 21 are used as inputs and Item X of the second information 31 is used as an output as illustrated in FIG. 14A, and a second determination/classification model 112B, in which Item a and Item b of the first information 21 are used as inputs and Item X of the second information 31 is used as an output as illustrated in FIG. 14B, are prepared in advance. In a case where the first information 21 is applied for Item a, Item b, and Item c, the first determination/classification model 112A is used. On the other hand, in a case where the first information 21 is applied for Item a and Item b (that is, in a case where Item c is omitted), the second determination/classification model 112B is used. In a case where the number of items of the first information 21 required for outputting the second information 31 is large (for example, 10 or more), the determination/classification model may be created by using the items selected based on a priority set for each item as training data. The priority may be defined based on, for example, a degree of contribution to the second information 31 in each item of the first information 21 or a probability of omission.

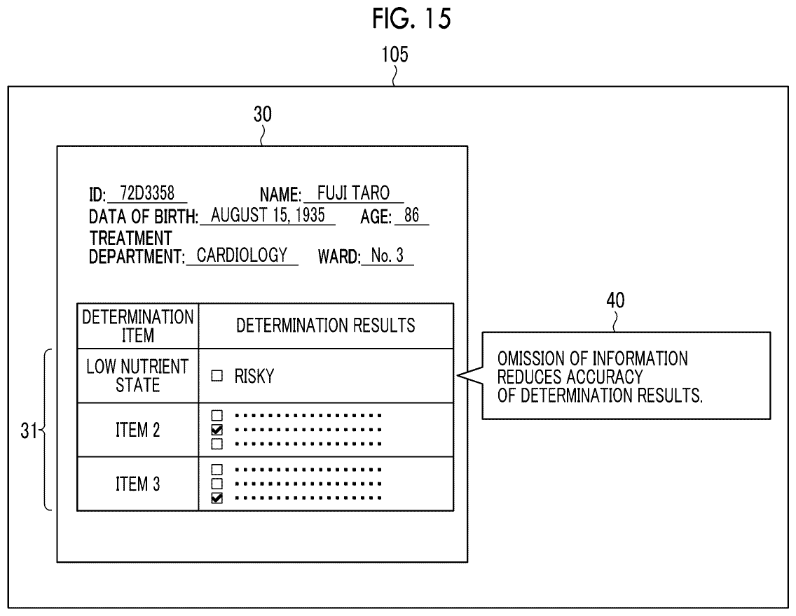

The presentation unit 15 performs a process of displaying the second medical document data 30, which includes the second information 31 derived based on a part of the first information 21 in which omission has not occurred, on the display 105 together with the omission information 40. Here, it is considered that accuracy of the second information 31 derived in a state in which the omission has occurred in the first information 21 is decreased compared to a case where omission has not occurred in the first information 21. Therefore, as illustrated in FIG. 15, the presentation unit 15 may display information, which indicates a fact that accuracy of the second information 31 that is derived based on a part of the first information 21 in which omission has not occurred is relatively low, on the display 105 as the omission information 40.

Further, the presentation unit 15 may display both the information (see FIG. 13) indicating the item of the first information 21 in which omission has occurred and the information (see FIG. 15) indicating a fact that accuracy of the second information 31 is relatively low, on the display 105 as the omission information 40.

Further, the presentation unit 15 may change a content of information to be displayed as the omission information 40 according to the accuracy of the second information 31 derived based on a part of the first information 21 in which omission has not occurred. For example, in a case where the accuracy of the second information 31 is less than a threshold value, information indicating the item of the first information 21 in which omission has occurred may be displayed on the display 105 as the omission information 40, and in a case where the accuracy of the second information 31 is equal to or greater than the threshold value, information indicating a fact that the accuracy of the second information 31 is relatively low may be displayed on the display 105 as the omission information 40. In this case, for example, for each item of the first information 21 required for deriving the second information 31, a score is applied in accordance with a degree of contribution, and the total sum of the scores, which is calculated for a part of the first information 21 in which omission has not occurred, or a value obtained by normalizing the scores may be applied as the accuracy of the second information 31. The second information 31, which is derived by using the first determination/classification model 112A, and the second information 31, which is derived by using the second determination/classification model 112B, may be displayed in different display aspects. For example, similar to the second information 31 and the third information 32 in FIG. 8, the background colors of target items may be different between the second information 31 derived by using the first determination/classification model 112A and the second information 31 derived by using the second determination/classification model 112B. Further, similar to the second information 31 and the third information 32 in FIG. 9, different icons may be presented between the second information 31 derived by using the first determination/classification model 112A and the second information 31 derived by using the second determination/classification model 112B. Further, similar to the field in which the first information 21 is presented in FIG. 10, a display format of the omission information 40 is not limited thereto. A display position of the omission information 40 is not limited as long as the correspondence relationship with the second information 31, which is derived by using the corresponding second determination/classification model 112B, is clear. For example, in a case where Item 1 is the second information 31 derived by using the second determination/classification model 112B, the omission information 40 corresponding to the second information 31 may be displayed between Item 1 and Item 2. Further, the omission information 40 may be always displayed in a case where the second medical document data 30 is presented, or the omission information 40 corresponding to the second information 31 may be displayed as a pop-up in a case where the second information 31 derived by using the second determination/classification model 112B is designated. The designation of the second information 31, which is derived by using the second determination/classification model 112B, may be performed by, for example, receiving an input from the user with respect to a display field of an item, an icon, or the like by the input device 104.

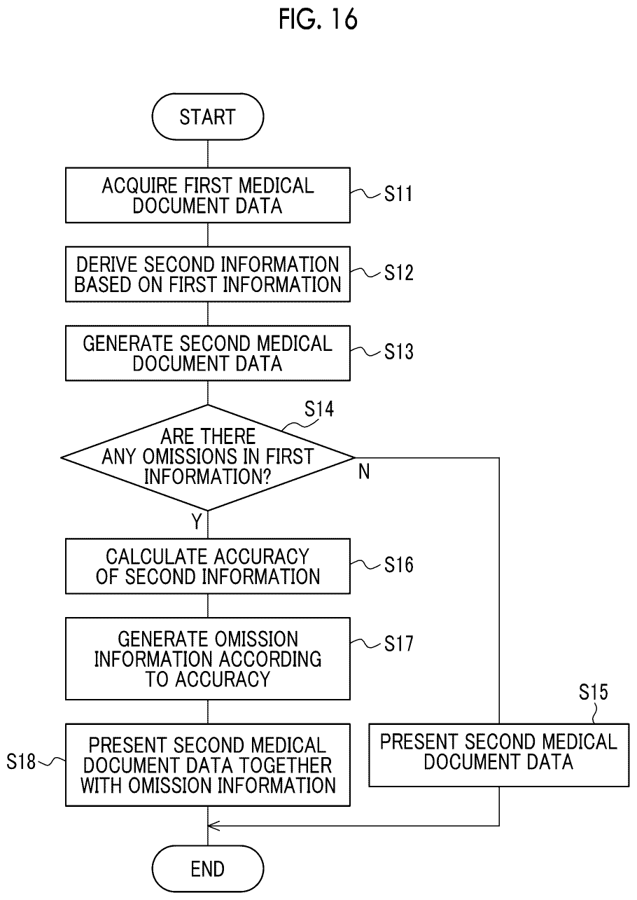

FIG. 16 is a flowchart showing an example of a flow of a process performed in the information processing apparatus 10 that has a function of changing the content of information displayed as the omission information 40 according to the accuracy of the second information 31 derived based on a part of the first information 21 in which omission has not occurred.

In step S11, the CPU 101 functions as the acquisition unit 11 and acquires the first medical document data 20 including the first information 21 related to the patient.

In step S12, the CPU 101 functions as the derivation unit 12 and derives the second information 31, which is different from the first information 21, related to the patient, based on the first information 21 included in the first medical document data 20 acquired in step S11. In a case where omission has occurred in a part of the plurality of pieces of first information 21 required for deriving the second information 31, the CPU 101 derives the second information 31 based on the other part of the first information 21 in which omission has not occurred. In step S13, the CPU 101 functions as the generation unit 14 and generates the second medical document data 30 including the second information 31 derived in step S12.

In step S14, the CPU 101 determines whether or not omission has occurred in the first information 21 in a case where the second information 31 is derived in step S12. In a case where the CPU 101 determines that omission has occurred in the first information 21, the CPU 101 functions as the presentation unit 15 in step S15 and displays the second medical document data 30, which is generated in step S13, on the display 105.

On the other hand, in a case where the CPU 101 determines that omission has occurred in the first information 21, in step S16, the CPU 101 calculates the accuracy of the second information 31 derived in step S12. In step S17, the CPU 101 generates the omission information 40 in accordance with the accuracy that is calculated in step S16. For example, the CPU 101 generates, in a case where the accuracy of the second information 31 is less than the threshold value, information indicating the item of the first information 21 in which omission has occurred as the omission information 40 and generates, in a case where the accuracy of the second information 31 is equal to or greater than the threshold value, information indicating a fact that the accuracy of the second information 31 is relatively low as the omission information 40.

In step S18, the CPU 101 functions as the presentation unit 15 and displays the second medical document data 30, which is generated in step S13, on the display 105 together with the omission information 40 generated in step S17.

Although the process related to the third information 32, which is recorded through a manual input, is omitted in the present flowchart, the second medical document data 30 may include the third information 32, and in this case, the second information 31 and the third information 32 may be displayed in an identifiable manner in the second medical document data 30.

As described above, the information processing apparatus 10 according to the present embodiment presents, in a case where omission has occurred in at least a part of the first information 21 required for deriving the second information 31 in the first medical document data 20, omission information 40, which indicates a fact that the omission has occurred, together with the second medical document data 30. According to the information processing apparatus 10 of the present embodiment, in a case where omission has occurred in the first information 21, it is possible to understand the meaning through the omission information 40. Further, by presenting the information, which indicates the item of the first information 21 in which omission has occurred, as the omission information 40, it is easy to determine the response with respect to the omission. Further, by presenting information, which indicates a fact that the accuracy of the second information 31 derived based on the other part of the first information 21 where omission has not occurred, as the omission information 40, it is possible to call attention to the second information 31 derived by allowing omissions.

Further, in the above embodiment, for example, as hardware structures of processing units that execute various processes, such as the acquisition unit 11, the derivation unit 12, the reception unit 13, the generation unit 14, and the presentation unit 15, various processors shown below can be used. The above-described various processors include, for example, a programmable logic device (PLD) which is a processor having a changeable circuit configuration after manufacturing, such as an FPGA, and a dedicated electrical circuit which is a processor having a dedicated circuit configuration designed to execute specific processing, such as an application specific integrated circuit (ASIC), in addition to the GPU and the CPU which is a general-purpose processor that executes software (programs) to function as various processing units, as described above.

One processing unit may be configured by one of the various processors or may be configured by a combination of the same or different kinds of two or more processors (for example, a combination of a plurality of FPGAs or a combination of the CPU and the FPGA). Further, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. A second example thereof is a form of using a processor that realizes the function of the entire system including the plurality of processing units by one integrated circuit (IC) chip, as represented by a system on chip (SoC) or the like. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

Moreover, in the above-described embodiment, an aspect has been described in which the document creation program 110 is stored (installed) in advance in the non-volatile memory 103, but the disclosed technology is not limited to this. The document creation program 110 may be provided in a form recorded in a recording medium such as a compact disc read only memory (CD-ROM), a digital versatile disc read only memory (DVD-ROM), and a universal serial bus (USB) memory. In addition, the document creation program 110 may be configured to be downloaded from an external device via a network. That is, the program (program product) described in the present embodiment may be provided by a recording medium or may be distributed from an external computer.

In addition, the disclosure of JP2021-169821 filed on Oct. 15, 2021 is incorporated herein by reference in its entirety. Further, all documents, patent applications, and technical standards described in the specification are incorporated herein by references to the same extent as the incorporation of the individual documents, patent applications, and technical standards by references are described specifically and individually.

What is claimed is:

1. An information processing apparatus comprising:
at least one processor,
wherein the processor is configured to:
acquire first medical document data including first information related to a patient;
derive second information, which is different from the first information related to the patient, based on the first information; and
generate second medical document data including the derived second information.

2. The information processing apparatus according to claim 1,
wherein the processor is configured to derive a result, which is obtained by performing determination or classification based on at least one piece of the first information, as the second information.

3. The information processing apparatus according to claim 1,
wherein the processor is configured to derive the second information based on a predetermined rule.

4. The information processing apparatus according to claim 1,
wherein the processor is configured to derive the second information by using a trained model that is trained through machine learning, which uses a plurality of combinations of the first information and the second information as training data, that uses the first information as an input, and that uses the second information as an output.

5. The information processing apparatus according to claim 1,
wherein the processor is configured to derive one piece of the second information based on a plurality of pieces of the first information.

6. The information processing apparatus according to claim 1,
wherein the processor is configured to present the second medical document data by associating the first information, which is used for deriving the second information, with the second information.

7. The information processing apparatus according to claim 1,
wherein the processor is configured to:
receive an input of third information related to the patient; and
generate the second medical document data in which the second information and the third information are recorded in an identifiable state from each other.

8. The information processing apparatus according to claim 1,
wherein the processor is configured to:
receive an input of third information related to the patient;
generate the second medical document data in which the second information and the third information are recorded; and
present the second medical document data such that the second information and the third information are made to be in an identifiable state.

9. The information processing apparatus according to claim 1,
wherein the processor is configured to present, in a case where omission has occurred in at least a part of the first information required for deriving the second information in the first medical document data, omission information, which indicates a fact that the omission has occurred, together with the second medical document data.

10. The information processing apparatus according to claim 9,
wherein the processor is configured to present information, which indicates an item of the first information in which the omission has occurred, as the omission information.

11. The information processing apparatus according to claim 9,
wherein the processor is configured to:
derive, in a case where omission has occurred in a part of a plurality of pieces of the first information required for deriving the second information, the second information based on another part of the first information in which omission has not occurred; and
present the second medical document data, which includes the derived second information, together with the omission information.

12. The information processing apparatus according to claim 11,
wherein the processor is configured to present information, which indicates a fact that accuracy of the second information derived based on the other part of the first information in which the omission has not occurred is relatively low, as the omission information.

13. The information processing apparatus according to claim 11,
wherein the processor is configured to present, in a case where accuracy of the second information derived based on the other part of the first information in which the omission has not occurred is less than a threshold value, information, which indicates an item of the first information in which the omission has occurred, as the omission information, and present, in a case where the accuracy is equal to or greater than the threshold value, information, which indicates a fact that the accuracy of the second information is relatively low, as the omission information.

14. The information processing apparatus according to claim 1,
wherein the first information is information to be recorded in a plurality of items included in the first medical document data, and
the second information is information to be recorded in at least one item included in the second medical document data.

15. The information processing apparatus according to claim 1,
wherein the first medical document data and the second medical document data are data obtained by converting documents, which are related to hospital admission and discharge of the patient, into data.

16. The information processing apparatus according to claim 1,
wherein the second information is information related to a necessity of nursing care or assistance for the patient.

17. An information processing method executed by at least one processor included in an information processing apparatus, the method comprising:
acquiring first medical document data including first information related to a patient;
deriving second information, which is different from the first information related to the patient, based on the first information; and
generating second medical document data including the derived second information.

18. A non-transitory computer-readable storage medium storing an information processing program causing at least one processor included in an information processing apparatus to execute a process comprising:
acquiring first medical document data including first information related to a patient;
deriving second information, which is different from the first information related to the patient, based on the first information; and
generating second medical document data including the derived second information.
